(19) United States
(12) Patent Application Publication
(10) Pub. No.: US 2017/0218483 A1
(43) Pub. Date: Aug. 3, 2017

(54) MEDICAL BIODEGRADABLE ZN-CU ALLOY AND ITS PREPARATION METHOD AS WELL AS APPLICATIONS

(71) Applicant: SHANGHAI JIAO TONG UNIVERSITY, Shanghai (CN)

(72) Inventors: Hua HUANG, Shanghai (CN); Guangyin YUAN, Shanghai (CN)

(21) Appl. No.: 15/485,773

(22) Filed: Apr. 12, 2017

Related U.S. Application Data
(63) Continuation of application No. PCT/CN2016/089732, filed on Jul. 12, 2016.

(30) Foreign Application Priority Data
Aug. 19, 2015 (CN) 201510512800.6

(51) Int. Cl. C22C 18/02; A61F 2/82; A61L 31/14; A61B 17/86; A61L 27/58; A61L 27/04; A61B 17/80; A61F 2/28; A61L 31/02

(52) U.S. Cl. CPC C22C 18/02; A61F 2/28; A61F 2/82; A61L 31/148; A61L 31/022; A61L 27/58; A61L 27/047; A61B 17/80; A61B 17/86; A61L 2430/02

(57) ABSTRACT

The present invention relates to the preparation methods and applications of biodegradable zinc-copper alloys, which can be applied to medical implant materials. The alloy of present invention is mainly composed of copper (1-10 wt. %), the balance of zinc and trace impurity elements. As-cast alloy ingot is homogenized and then hot processed to refine microstructure. The mechanical properties of the alloys are improved due to the refined microstructure. The alloys are capable of being further fabricated into micro-tubes, wires and plates. There are many advantages of these Zn alloys such as excellent mechanical properties, easy to process, appropriate corrosion resistance, good biocompatibility and so on, which correspondingly can be applied to many kinds of biodegradable medical implant devices. With excellent mechanical properties, good biocompatibility and degrading completely in 6-18 months, the Zn alloys meet the requirements of implant materials for mechanical properties and biosafety.

### TECHNOLOGY FIELD

The present invention relates to the preparation methods and applications of the medical biodegradable zinc-copper alloys, which belong to technology field of medical material.

### TECHNOLOGY BACKGROUND

At present, non-degradable metal materials, such as austenitic stainless steel, cobalt-chromium alloy, tantalum-titanium and its alloys, nickel-titanium shape memory alloys, and platinum-iridium alloys, are generally applied as medical materials implanted into human body. The applications of these permanent implant materials have following drawbacks. For example, after being implanted into human body, it is easy for the permanent metal stents to form thrombosis caused by in-stent restenosis and endothelial dysfunction because of their non-biodegradability. Stent, permanently retained in the body, cannot be removed, which will make it difficult to re-implant a vascular stent again if the vascular occlusion happens at the same place. Another example is that when bone screws and plates of stainless steel and titanium alloys are implanted in human body, it is necessary to remove the implants by secondary surgery after the bone tissue heals, which will result in an increase of pain and financial burden for patients. Therefore, study and development of biomedical metal materials with good mechanical properties, biocompatibilities and in vivo degradability has become an important development direction in this field.

### SUMMARY OF THE INVENTION

The object of present invention is to provide novel medical biodegradable Zn—Cu alloys as well as their preparation methods and applications against the drawbacks of present technology.

Present invention is implemented by the following technical solutions:

The first aspect, present invention provides biodegradable Zn—Cu alloys for medical application, which are composed of copper, zinc and unavoidable impurity elements, of which the content of copper is 1-10 wt. % and the content of impurity elements is no more than 0.1 wt. %.

As a preferable solution, content of copper is 1-4 wt. %.

The second aspect, present invention provides preparation methods of biodegradable Zn—Cu alloys for medical application. Herein, Raw materials are pure zinc and brass or pure zinc and pure copper, which are melted and then cast to obtain Zn—Cu alloy ingot. Preferably, the purity of pure zinc is not less than 99.995%, the content of copper in brass is 62 wt. % and the purity of copper is not less than 99.99%.

As a preferable solution, the preparation methods of the biodegradable Zn—Cu alloys for medical application include the following preparation process:

At first, brass or pure copper is added after pure zinc is heated and melted completely. When brass or pure copper is melted completely, the melt of the patent alloy is obtained.

The second, after stirring the melt, removing slag, standing for several minutes, and casting, the as-cast alloy ingot is obtained.

The third, after the as-cast alloy is homogenized at about 350-380° C., the hot extrusion is performed at 200˜350° C. Followed by drawing or rolling, rods, wires tubes and plates can be obtained.

As a preferable solution, the extrusion ratio of the hot extrusion process described above is controlled between 9 and 50.

As a preferable solution, the amount of deformation of the rolling process is controlled between 5 and 20%.

The third aspect, present invention provides the applications of novel medical biodegradable Zn—Cu alloys on medical devices.

As a preferable solution, the medical devices described above include tube-type devices and one of the devices of bone implants.

As a preferable solution, the described tube-type devices include vascular stent, bile duct stent or tracheal stent and one of the devices of bone implants include bone plates or bone screws.

As is well known, copper and zinc are one of the most essential trace elements in human body. Among them, zinc can promote cell renewal, enhance the body immunity, and maintain growth and development of body. Zinc is one of the components of many proteins and nucleic acid synthases, and is the active center of hundreds of enzymes. Zinc is also a component of insulin, which is the key factor to maintain normal life activities. Zinc deficiency may lead to all physiological dysfunction of human body. Zinc alloys as potential biodegradable medical implant materials have promising application prospects.

Copper accounts for one millionth of human body weight and each human contains copper about 100˜150 mg. The daily intake of copper for an adult is 3˜5 mg to maintain the metabolic balance. The main symptoms of copper deficiency for infants and girls are neutropenia, hypochromic anemia, osteoporosis and osteopenia similar to bone lesions caused by vitamin C deficiency, which can't heal using iron treatment. Besides, copper deficiency can also cause pigment reduction of skin and hair, seborrheic dermatitis, superficial vein expansion, anorexia, diarrhea, hepatosplenomegaly and retarded growth. The physiological functions of copper are mainly shown in the following several aspects. 1. Copper can contribute to maintaining normal hematopoietic function including promoting absorption and transport of iron and promoting synthesis of hemoglobin and hemoglobin because of the formation ceruloplasmin. 2. Copper can contribute to maintaining normal bones, blood vessels and skin. Copper Lysyl Oxidase can promote cross-linking between collagen and elastin of bone, blood vessels and skin. 3. Copper can also keep the central nervous system healthy. 4. Copper can protect the body cells from being poisoned by superoxide. 5. Copper ion can induce endothelial growth factor, promote endothelial proliferation, accelerate the process of vascular regeneration, and prevent excessive proliferation of smooth muscle cells. Copper can inhibit thrombosis and reduce rate of in-stent restenosis effectively. (Reference: G. f. Hu. Copper stimulates proliferation of human endothelial cells under culture [J]. Journal of Cellular Biochemistry. 1998,69(3):326-335.). 6. Copper ion can promote new bone formation and growth by promoting collagen deposition, which means copper can promote osteogenesis. (Reference: C. Gérard, L. -J. Bordeleau, J. Barralet, C. J. Doillon. The stimulation of angiogenesis and collagen deposition by copper[J]. Biomaterials. 2010,31(5):824-831.). 7. Copper ions have bactericidal property, which is important for medical implant devices. 8. Other effects: Copper has effects on cholesterol metabolism, oxidative metabolism of myocardial cells, host defense, hormone excretion and many other physiological, biochemical and pathophysiological processes. Thus, copper is chosen as main alloying element for Zn-based alloys considering its improvement effect of both strength and elongation. On the other hand, during degradation of the implant, the release of trace Cu ions has beneficial biological functions described above for human body. This is the innovative design idea of biodegradable Zn—Cu binary alloy in this invention.

### Advantageous Effects of Invention

Compared with current technologies, present invention has the following advantages:

1. The Zn alloys in present invention exhibit proper mechanical properties, easy processing property, good corrosion resistance, good biocompatibility and many other advantages. The yield strength (YS), ultimate tensile strength (UTS) and elongation values of the Zn alloys can reach 150˜230 MPa, 187˜271 MPa and 22.2˜52.3%, respectively.

2. The Zn alloys in present invention exhibit good corrosion resistance, whose corrosion rate measured in hank's solution at 37° C. is about 0.02˜0.2 mm year⁻¹.

3. The Zn alloys in present invention can be applied as materials for fabricating many kinds of biodegradable medical implants, which have proper mechanical properties as well as good biocompatibility and will degrade completely within 6-18 months.

4. The Zn alloys in present invention are proper for fabricating biodegradable medical wires, vascular stents, bile duct stents, tracheal stents, bone plates, bone screws, bone tissue engineering scaffolds and so on, which meet the requirements of medical devices described above for mechanical properties and biosafety.

### BRIEF DESCRIPTION OF DRAWINGS

Other features, objects, and advantages of the present invention will become more apparent by reading and referring the detailed description of nonrestrictive embodiment in following figures.

FIG. 1 shows the microstructures of four as-cast alloys described in embodiment one;

FIG. 2 shows the microstructures of four as-extruded alloys described in embodiment two.

### DESCRIPTION OF EMBODIMENT

Next, present invention is illustrated in detail combined with specific embodiments. The following embodiments will assist one skilled in this technology to further understand the invention, but not to limit the invention in any way. It should be noted that several modifications and improvements can be made by one skilled in this technology without deviating from the conception of present invention. All these are within the scope of present invention.

The biodegradable medical Zn—Cu alloys of present invention contain Cu of 1˜4 wt. % and the balance of Zn.

Properties of material depend on the microstructure of it. Thus, the microstructure of material can be modified according to application requirements. For example, microstructure of alloys can be modified to improve the properties of alloys by modifying the composition of the alloys within the composition range described above, homogenizing the alloys, processing the alloys (e.g. rolling and extrusion).

### Embodiment 1

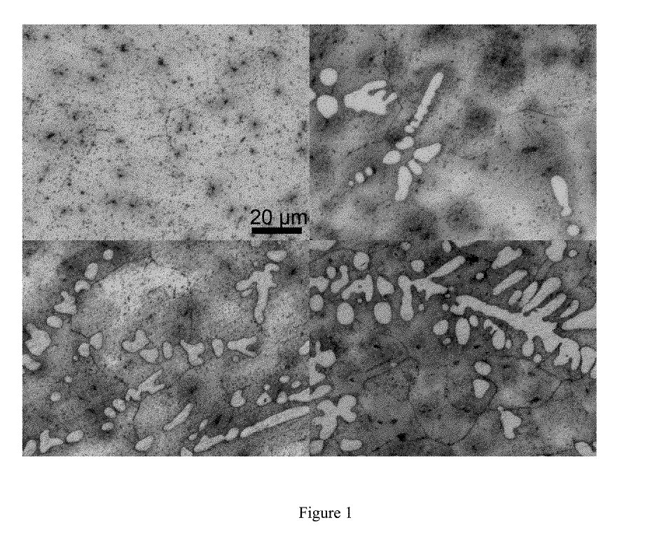

Zn—Cu alloy ingots are obtained by using current traditional melting and casting process of electric resistance furnace. Brass or pure copper is added and melting is kept at 550° C. for one hour until brass or pure copper is melted completely after pure zinc is heated and melted completely in electric resistance furnace. After stirring the melt for 10˜15 minutes, removing slag, standing for 10˜30 minutes, and casting, as-cast Zn—Cu binary alloy ingots are then obtained. Microstructure of four representative Zn-xCu (x=1, 2, 3, 4 wt. %) alloys is shown in FIG. 1.Raw materials are pure zinc (99.995%) and brass (Cu-38wt. %Zn) or pure copper (99.99%). The secondary phase of four binary alloys described above is CuZn₅ phase (white dendritic secondary phase), as shown in FIG. 1. Biocompatibility test indicates that all the four Zn—Cu binary alloys described above exhibit good biocompatibility without obvious cytotoxicity.

### Embodiment 2

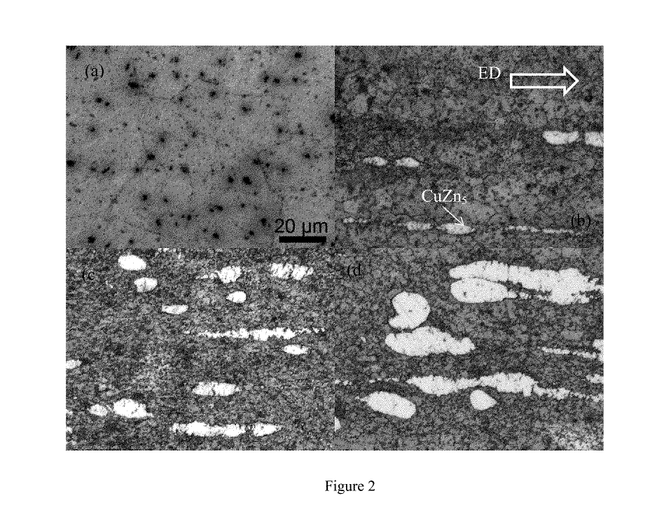

Four representative Zn-xCu (x=1, 2, 3, 4 wt. %) alloys described in embodiment one are homogenized at 360° C.˜380° C. for 8 hours. And then the alloys are extruded at 280° C. with extrusion ratio of 9:1, as a result of which more uniform and finer microstructure of as-extruded rods or plates can be obtained. In this way, the properties of the alloys are improved. Microstructure of as-extruded alloys is shown in FIG. 2. After extrusion, the secondary phase is crushed, lengthened and distributes along the extrusion direction. Grain size of as-extruded alloys is refined of 1˜10 μm. The results of tensile test in room temperature are shown in Table 1. The yield strength (YS), ultimate tensile strength (UTS) and elongation values of the Zn—Cu binary alloys can reach 150˜230 MPa, 187˜271 MPa and 22˜55%, respectively. The corrosion rate of Zn—Cu binary alloys measured in hank's solution at 37° C. is about 0.02˜0.2 mm year⁻¹. These properties meet the requirements of biodegradable medical materials for clinical application and Zn—Cu binary alloys are proper for fabricating biodegradable medical implants including wires, vascular stents, bile duct stents, tracheal stents, bone plates, bone screws, bone tissue engineering scaffolds and so on.

The mechanical properties of the Zn-xCu (x=1, 2, 3, 4 wt. %) alloys described in this embodiment are shown in Table 1.

TABLE 1
Mechanical properties of as-extruded alloys

| alloys | UTS (MPa) | YS (MPa) | Elongation (%) |
|---|---|---|---|
| Zn-1 wt. % Cu | 187 | 150 | 22.2 |
| Zn-2 wt. % Cu | 241 | 204 | 47.4 |
| Zn-3 wt. % Cu | 258 | 215 | 49.1 |
| Zn-4 wt. % Cu | 271 | 230 | 52.3 |

### Embodiment 3

Zn-1Cu alloy described in embodiment one is machined into plate of 10 mm in thickness and then rolled at 350° C., of which the amount of deformation of each rolling pass is about 10%. At last, plate of 2 mm in thickness can be obtained. The UTS, YS and elongation values of plates along rolling direction are 210 MPa, 160 MPa and 19.8%, respectively. The corrosion rate of this plate measured in hank's solution at 37° C. is about 0.18 mm year⁻¹. These properties meet the requirements of biodegradable medical materials for clinical application and Zn-1Cu binary alloys are proper for fabricating bone implants like bone plates.

### Embodiment 4

As-extruded rods of Zn-2Cu alloy described in embodiment two are machined into tubes of 20 mm in diameter and 13 mm in height. After being extruded at 300° C., seamless tube of 8mm in outer diameter and 0.8 mm in thickness of wall can be obtained. The seamless tubes are drawn or rolled in room temperature. After annealing treatment of 300° C. for 30 min is performed during each rolling or drawing pass, micro-tube of 3 mm in outer diameter and 0.185 mm in thickness of wall. The UTS, YS and elongation values of the tubes are 240 MPa, 200 MPa and 40.3%, respectively. The corrosion rate of this plate measured in hank's solution at 37° C. is about 0.12 mm year⁻¹. These properties meet the requirements of biodegradable medical stents for clinical application and Zn-2Cu binary alloys are proper for fabricating vascular stents, bile duct stents and tracheal stents.

### Embodiment 5

As-extruded rods of Zn-2Cu alloy described in embodiment two are machined into cylinder of 20 mm in diameter and 30 mm in height. After being extruded at 330° C., wires of 1.5 mm in diameter can be obtained. The UTS, YS and elongation values of the wires are 270 MPa, 220 MPa and 52%, respectively. The seamless tubes are drawn in room temperature for many passes. After annealing treatment of 350° C. for 30 min is performed during each drawing pass, micro-wires of 500 mm-50 μm in diameter can be obtained. The typical UTS, YS and elongation values of the wires are 255 MPa, 216 MPa and 44.6%, respectively. The corrosion rate of the wires measured in hank's solution at 37° C. is about 0.08 mm year⁻¹. Biodegradable catheters weaved by these wires can be applied to stents for implantation including vascular stents, bile duct stents, tracheal stents and so on.

The specific embodiments of the present invention have been described above. It should be noted that the invention is not limited to the specific embodiments described above and various changes or modifications may be made by one skilled in this technology within the scope of the claims but this does not affect the substantial content of present invention.

1. Biodegradable Zn—Cu alloys for medical application, wherein the alloys are composed of copper, zinc and unavoidable impurity elements, of which the content of copper is 1-10 wt. % and the content of impurity elements is no more than 0.1 wt. %.

2. The Biodegradable Zn—Cu alloys for medical application as claimed in claim 1, wherein the preferably content of copper is 1-4 wt. %.

3. A preparation method of biodegradable Zn—Cu alloys for medical application as claimed in claim 1, wherein the Zn—Cu ingot is prepared by using pure zinc and brass or by using pure zinc and pure copper.

4. The preparation method as claimed in claim 3, wherein the method comprises the following steps:
At first, brass or pure copper is added when pure zinc is heated and melted completely. When brass or pure copper is melted completely, the melt of the patent alloy is obtained.
The second, after stirring the melt, removing slag, standing for several minutes, casting, the as-cast alloy ingot is obtained.
The third, after the as-cast alloy is homogenized at about 360-380° C. for 8-10 hours, the hot extrusion is performed at 200˜350° C. Followed by drawing or rolling, rods, wires, tubes and plates can be obtained.

5. The preparation method as claimed in claim 4, wherein the biodegradable Zn—Cu alloys for medical application are characterized by the characteristic that the extrusion ratio of the hot extrusion process described above is controlled between 9 and 50.

6. The preparation method as claimed in claim 4, wherein the biodegradable Zn—Cu alloys for medical application are characterized by the characteristic that the amount of deformation of the rolling process is controlled between 5 and 20%.

7. The applications of biodegradable Zn—Cu alloys for medical application as claimed in claim 1, wherein the biodegradable Zn—Cu alloys are used in medical application. The applications as claimed in claim 7, wherein the biodegradable Zn—Cu alloys are used in medical devices including tube-type devices and one of the devices of bone implants.

8. The applications as claimed in claim 8, wherein the tube-type devices include vascular stent, bile duct stent or tracheal stent and one of the devices of bone implants include bone plates or bone screws.
